(19) United States
(12) Patent Application Publication
(10) Pub. No.: US 2006/0100618 A1
(43) Pub. Date: May 11, 2006

(54) System and method for performing ablation and other medical procedures using an electrode array with flex circuit

(75) Inventors: Eric K.Y. Chan, San Carlos, CA (US); Gabriel Vegh, Huntsville, UT (US)

Correspondence Address: FITCH EVEN TABIN AND FLANNERY, 120 SOUTH LA SALLE STREET, SUITE 1600, CHICAGO, IL, 60603-3406, US

(73) Assignee: Cardima, Inc.

(21) Appl. No.: 11/268,941

(22) Filed: Nov. 8, 2005

Related U.S. Application Data
(60) Provisional application No. 60/625,859, filed on Nov. 8, 2004.

(51) Int. Cl. A61B 18/14

(52) U.S. Cl. 606/41

(57) ABSTRACT

An ablation catheter having distal and proximal ends for performing ablation on a human tissue region comprises at least one electrode. These elements are formed on a conductive sheet situated at the distal end of the catheter. A flex circuit assembly couples the at least one electrode to a measurement and power circuit attached to the proximal end of the catheter. The measurement and power circuit supplies power to the at least one electrode via the flex circuit.

### FIELD OF THE INVENTION

The invention relates to catheters and other medical probes and, more specifically, to using flex circuits and etched electrodes in these devices.

### BACKGROUND OF THE INVENTION

Certain catheters or surgical probe shafts employ a set of braided insulated copper wires that form an intertwined, complicated cross-hatched design running the length of the catheter or probe. This braided shaft then serves as a conduit for radio frequency (RF) current that is delivered to the electrodes to ablate tissue, as well as to sense electrophysiological signals that are in turn transmitted along those same lines to a monitoring system.

Another pair of copper wires is often soldered to a copper-constantan thermocouple junction located on a gold band proximal to each electrode. This gold band has a high thermal conductivity and the thermocouple junction quickly equilibrates to the sensed environmental temperature at the gold band. The thermocouple junction forms a temperature-to-voltage transducer and the two copper wires transmit information back to the energy source for feedback-control of RF energy delivery.

Material and labor costs may increase in the assembly process as the number of electrodes increases with conventional methods of assembly. For example, the number of braided wires for a 24-electrode catheter/probe with 24 thermocouples adds up to 72 wires. The “count and cut” process used during assembly to extricate and expose the correct wire along the shaft to solder onto an electrode or thermocouple has become increasingly time-consuming to perform these labor-intensive production steps. When one electrode or one thermocouple connection fails during final electrical testing at the factory, the entire catheter/probe has to be counted as scrap if the fault cannot be reworked.

### SUMMARY OF THE INVENTION

An ablation catheter having etched electrodes connected to the proximal end of the catheter by a flex circuit enables the braided wire assembly used in previous systems to be replaced by printed circuit board technology. Thermal sensing elements (e.g., thermocouples or thermistors) may also be connected. The catheter is easy to fabricate because of the use of the flex circuits in conjunction with etched electrodes and thermal sensing elements such as thermocouples. The use of etching to construct the electrodes allows electrodes having very precise dimensions to be constructed. Alternatively, coiled electrodes can be used.

In many of these embodiments, a catheter having distal and proximal ends for performing ablation on a human tissue region comprises at least one etched electrode. In addition, at least one thermal sensing element may be used. These elements are formed from a conductive sheet and situated at the distal end of the catheter. Alternatively, coiled electrodes can be used.

A flex circuit assembly couples the at least one etched electrode and the at least one thermocouple sensor to a measurement and power circuit attached to the proximal end of the catheter. The measurement and power circuit supplies power, senses impedance at the electrode-tissue interface and controls electrical current flow to the at least one etched electrode via the flex circuit. The thermal sensing element supplies thermal information indicative of conditions at the human tissue interface to the measurement and power circuit, to control the amount of electrical current to be delivered to the tissue.

The flex circuit assembly may include plurality of identical flex circuit sub-portions. The sub-portions may be attached together and bent to form a cylindrically shaped assembly. Furthermore, multiple layers of flex circuits may be used. In addition, the measurement and power circuit may be comprised of a PC board, an energy source, and monitoring equipment (e.g., monitoring and control circuits for energy delivery).

The etched electrodes may be coated with a conductive gel to aid in the ablation or other medical procedure. Also, the electrodes may be infused with anti-coagulant chemicals that are time released during the course of an ablation procedure. Further, the thermal sensing element may be comprised of gold bands and copper-constantan junctions. Mass production time and costs are reduced.

Thus, the present system and method allows for the replacement of complex braided wire arrangements with a flex circuit arrangement. The structures described herein are simple to construct and easy to modify when adjustments are needed and/or when failures of components occur after the flex circuit assembly is placed inside a catheter shaft.

In addition, the approaches described herein are useful in a variety of medical therapy applications. For instance, the embodiments described herein can also be employed for the treatment of cardiac arrhythmias such as atrial fibrillation (AF) and ventricular tachycardia (VT). Minimally invasive access or endocardial access methods can be employed with probes/catheters using these approaches. The electrodes described herein can also be used to sense electrical activity from the heart, and the proximal connection of the probe/catheter shaft can be attached to a computerized mapping system. In addition, the present approaches are useful in other tissue desiccation and ablation procedures, for example, in oncology to selectively heat and destroy cancerous tumors. Other uses in different organ systems are possible.

### BRIEF DESCRIPTION OF THE DRAWINGS

FIG. 1a is perspective view of a flex circuit assembly for use in an ablation catheter showing a single electrode thermocouple pair according to the present invention;

FIG. 1b is a front view of a flex circuit of FIG. 1 showing twenty four electrode-thermocouple pairings according to the present invention;

FIG. 1c is a perspective view showing a three-layered flex circuit assembly according to the present invention;

FIG. 2a is a perspective view of a flex circuit assembly with etched electrodes and thermocouples formed into a cylinder according to the present invention;

FIG. 2b is a perspective view of a flex circuit assembly with coiled electrodes and thermocouples formed into a cylinder according to the present invention;

FIG. 3a is a perspective view of a flex circuit assembly with etched electrodes and thermocouples formed into a cylinder according to the present invention;

FIG. 3b is a perspective view of a flex circuit assembly with coiled electrodes and thermocouples formed into a cylinder according to the present invention;

FIG. 4 is a perspective view of a flex circuit assembly fitted into an ablation catheter according to the present invention; and

FIG. 5 is a cross-sectional view taken along line 304 of FIG. 3a according to the present invention;

FIGS. 6a-c are cross-sectional views of a catheter using three flex circuit layers according to the present invention;

FIG. 7 is a perspective view of the catheter using three flex circuit layers of FIG. 6 according to the present invention; and

FIG. 8 is perspective view of a flex circuit sheet showing the electrodes etched directly onto a conductive sheet according to the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful in a commercially feasible embodiment are often not depicted in order to facilitate a less obstructed view of the various embodiments of the present invention.

### DETAILED DESCRIPTION OF THE PREFERRED EMBODIMENT

The present system and method allows for the replacement of complex braided wire arrangements with a flex circuit arrangement in catheters and other medical devices. Medical devices constructed according to these approaches are relatively simple to fabricate. Mass production time and costs are also reduced.

The approaches described herein can be used in a variety of medical procedures. For example, the approaches described herein can be employed for the treatment of cardiac arrhythmias such as atrial fibrillation (AF) and ventricular tachycardia (VT). Minimally invasive access or endocardial access methods can also be performed with the probes/catheters described in this application. The electrodes utilized in the approaches described herein can also be used to sense electrical activity from the heart, and the proximal connection of the probe/catheter shaft can be attached to a computerized mapping system. In addition, these approaches can be used in tissue desiccation and ablation procedures, for example, in oncology, to selectively destroy cancerous tumors.

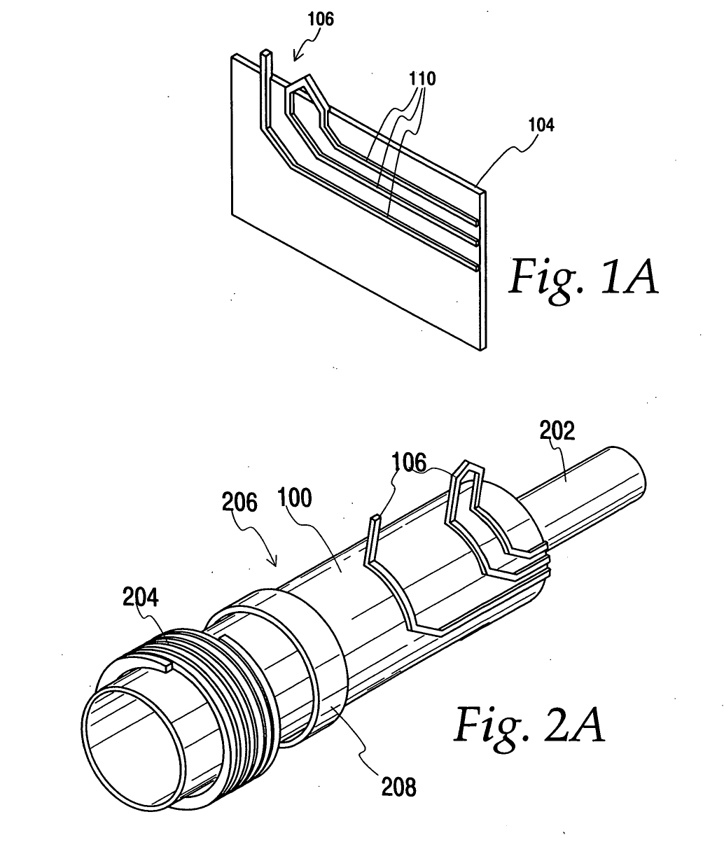

Referring now to FIG. 1a, one example of a flex circuit 100 used in an ablation catheter is described. A flex circuit pattern is printed on a flat sheet 104 with solder pins 106 at one edge of the sheet 104. The pins 106 point perpendicular to the surface of the sheet 104. The pins 106 correspond to connections for electrodes and thermal sensing elements (e.g., thermocouples). The pins are shown in FIG. 1 as being parallel to the surface of the sheet 104, but are bent or formed perpendicular to the sheet when the sheet is folded into a cylinder. The following description is made with respect to the thermal sensing elements being thermocouples. However, it will be understood by those skilled in the art that the thermal sensing elements may include not only thermocouples, but thermistors or any other thermal sensing device.

A conductive circuit 110 is established on the pattern and is connected to the pins 106. For example, a metallic conductive circuit 110 is established using techniques that are known in the art. In this case, the conductive circuit 110 includes three lines that conduct electrical energy.

In addition, as described with respect to FIGS. 1b and 1c, repeated similar patterns of the conductive circuits can be printed onto flex circuit boards. In addition, as described below, this arrangement can be formed into a cylinder and placed into the shaft of a catheter or medical probe.

Conducting circuit elements 110 of the sheet 104 are electrically insulated from each other and from the exposed surfaces of the flex sheet 104. Preferably, the inter-wire spacings for RF energy and current delivery are predetermined to comply with applicable regulatory, EMC and safety compliance standards.

Referring now to FIG. 1b, a circuit including 24 electrode and thermocouple pairs is shown. A first electrode thermocouple pair 106 (electrode E1 and thermocouple TC1) has corresponding conductive paths 110, which couple the electrode and thermocouples to a connector 150 at the proximal end of the catheter. A second electrode thermocouple pair 120 (electrode E2 and thermocouple TC2) has corresponding conductive paths 112, which couple the electrode and thermocouples to the connector 150 at the proximal end of the catheter. A third electrode thermocouple pair 122 (electrode E3 and thermocouple TC3) has corresponding conductive paths 114, which couple the electrode and thermocouples to the connector 150 at the proximal end of the catheter. For simplicity, the fourth through twenty-third pairs of electrodes and thermocouples are not shown in FIG. 1b. Finally, a twenty-fourth electrode thermocouple pair 124 (electrode E24 and thermocouple TC24) has corresponding conductive paths 116, which couple the electrode and thermocouples to the connector 150 at the proximal end of the catheter.

It will be understood that the electrode thermocouple pairs and their conductive paths can be split across multiple layers of circuit boards. In other words, the first eight pairs may be placed on a first flex circuit board, the second eight pairings on a second flex circuit board, and the third eight pairings placed on a third flex circuit board. The three boards are stacked onto each other and then formed into a cylinder. Preferably, the three groupings are offset lengthwise from each other when the three layers are rolled into a cylinder for placement in the catheter.

Referring now to FIG. 1c, a multi-layered flex circuit assembly is described. A first assembly 180, second assembly 182, and third assembly 184 are formed into concentric cylinders with assembly 180 being the outermost protective layer assembly. Assembly 182 is inside assembly 180 and assembly 184 is inside assemblies 180 and 182. Electrode solder points E1, E2, and E3 are formed on the assembly 180. Other electrode solder points up to and including electrodes En are formed on the other assemblies. The assemblies 180, 182, and 184 are electrically insulated from each other by homogenous polyimide material layers (not shown in FIG. 1c) that are typically used in multi-layer flex circuit boards.

In addition, thermocouple solder points T1, T2 and T3 are formed on assembly 180. Other thermocouple solder points up to and including Tn are formed on the assemblies 182 and 184. Conductive lines 186 are coupled to the respective electrodes and thermocouples. The electrodes and thermocouples are attached to the actual solder points.

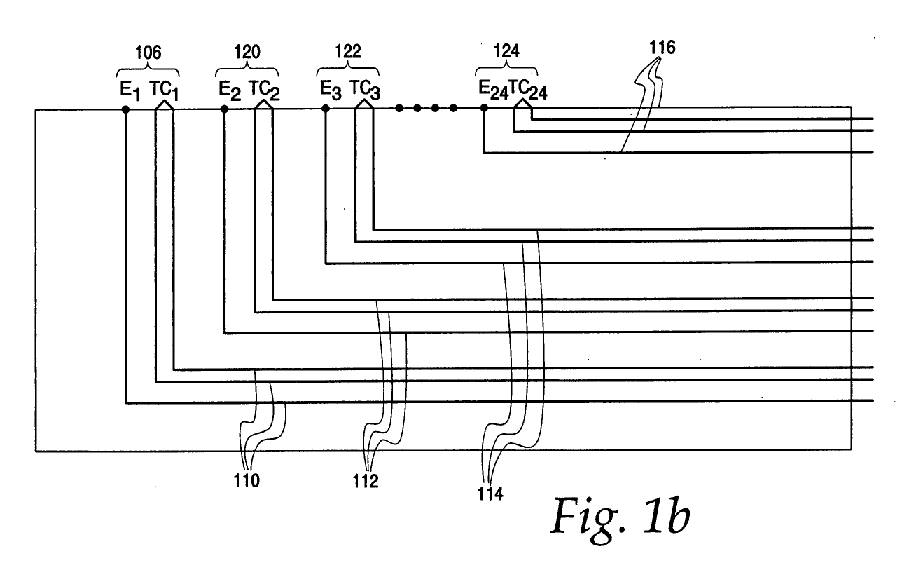

Referring now to FIG. 2a, the flex sheet 100 is shown folded into a cylinder 206. For example, the flex sheet 100 may be folded around a shape-forming mandrel 202, with the pins 106 at the sheet edge pointing away from the mandrel 202. In this case, the underside of the edge of the flex sheet 100 with pins 106 is adhered to the top surface of the other edge of the same sheet 100, so that the sheet takes on a cylindrical form. The pins 106 are soldered onto etched electrodes 204. The pins 106 (shown exaggerated in FIG. 2a for clarity) protrude perpendicularly along one longitudinal edge of the cylinder 206.

A thermocouple band 208 is also constructed. In one example, the thermocouple band 208 may be constructed of a gold band to give the band a high thermal conductivity. These bands can be constructed using techniques known by those skilled in the art.

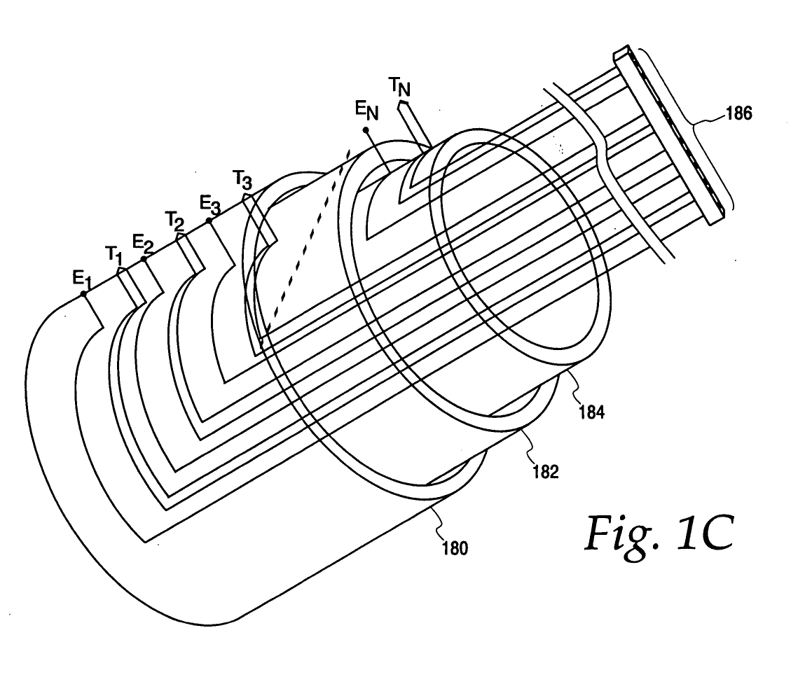

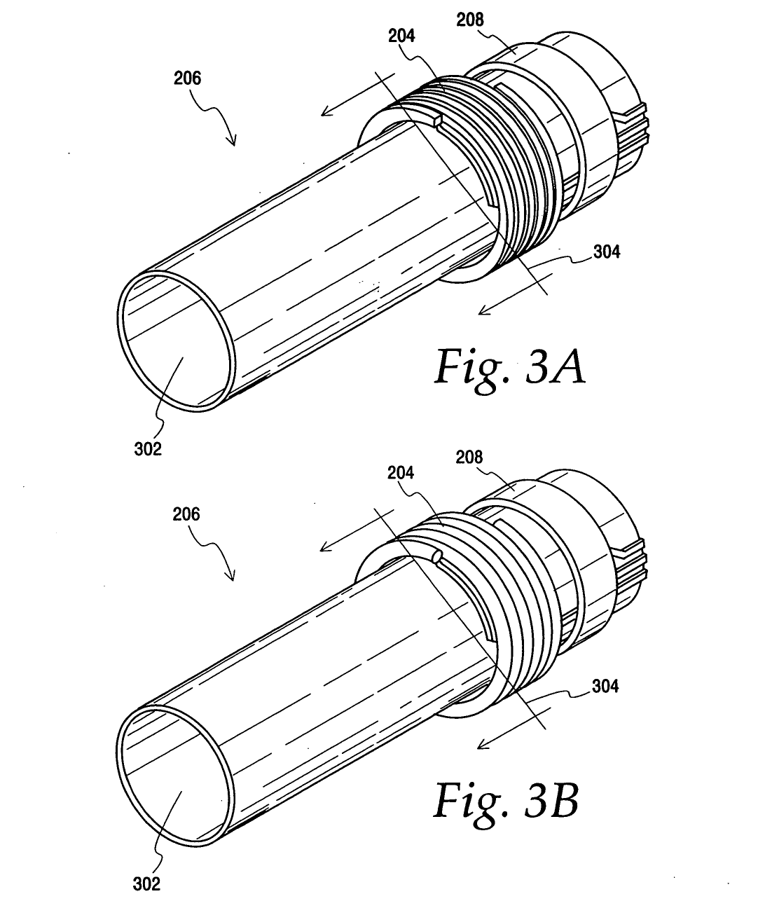

The example described herein with respect to FIG. 2a (and also FIGS. 3a and 5) utilizes a single set of electrodes and thermocouple band. However, multiple electrodes and bands can also be used. It will also be understood that multiples of the unit assembly can be organized in a linear pattern to form a linear mapping and ablation electrode array.

Preferably, metal etching is used for the production of the electrodes 204 to produce coiled groove, thereby creating a spring-like electrode component. Several techniques may be employed to etch metal sheets into different structural forms.

In one example process, a computer-aided design (CAD) drawing of the electrode coil pattern is generated. This drawing serves as the CAD image that is a faithful replica of the electrode. The drawing is printed onto a transparency film.

A cylindrical section of metal (e.g. platinum iridium) cut to a specific length is cleaned thoroughly. Then, a photo resist coating is applied to the outer surface so that it is photo-sensitive.

The CAD image is then overlaid onto the photo-sensitized metal surface and exposed to a ultra-violet (UV) light source. The metal cylinder is thereafter deposited into a developing solution to create a hardened image of the desired coil pattern on the metal cylinder surface.

The metal surface is then treated with an etchant, such as an acid. The etchant eats away the rest of the surface that is devoid of the hardened image, to create a spiral-shaped coil structure that can function as ablation and mapping electrodes 204. If the desired spiral groove is too fine for acid or other form of chemical etching, then an alternate fabrication technique is to employ three dimensional etching of the spiral pattern via a precision laser cutting process.

Yet another alternate process is to etch the electrodes directly onto the flex circuit board. This approach assumes dissimilar metals are layered onto the board, e.g. platinum for electrodes, copper for conduction lines by an appropriate manufacturing process.

Referring now to FIG. 2b, another example of a flex circuit assembly is described. In this case, the assembly is the same as that shown and described with respect to FIG. 2a except that the etched electrodes 204 are replaced with coiled electrodes 204.

In one example, the coiled electrodes 204 may be 0.005″ gauge (0.003″ to 0.006″ range with one preferred type being a 0.005″ gauge) platinum iridium wire that is wound into a spring-like structural unit. These units may be 3 mm to 6 mm long and have outer diameters ranging from approximately 3 Fr to 5 Fr. Other dimensions are also possible.

Referring now to FIG. 3a, the etched electrodes 204 and thermocouple band 208 are inserted over the cylindrical structure formed by folding the flex circuit. The electrodes 204 and thermocouple 208 are soldered at the respective protruding pin sites 106 that were spaced out by design to provide the desired inter-electrode and electrode-thermocouple spacing.

At one stage of the manufacturing process, the electrodes 204 can be coated with a conductive gel or other ionic material that improves tissue-electrode contact. At the same time, the electrodes 204 may be infused with anti-coagulant chemicals that are time released during the course of an ablation procedure.

Multiple layers of such unit assemblies may be utilized to reduce overall catheter or medical probe shaft diameter. These layers can be electrically insulated from each other by a homogenous polyimide material that is typically used in multi-layer flex circuit boards.

An inner hollow shaft 302 of the resulting cylinder from this flex circuit catheter shaft can serve as a conduit for a guide wire or stylet with deflectable mechanism, permitting the linear assembly of electrodes 204 and thermocouples 208 to be shaped and conformed to a tissue surface to afford excellent electrode-tissue contact that ensures optimal coupling of RF energy with the tissue. The conductive annular gold band for the thermocouple and the etched electrode are then slid along the shaft and soldered over their respective solder points.

The flex circuit assembly is rolled and placed in the shaft of the catheter. The end of the flex circuit assembly plugs into a connector. The connector is coupled to at least one PC card, which interfaces the arrangement to power and measurement equipment.

Referring now to FIG. 3b, another example of a flex circuit assembly is described. In this case, the assembly is the same as that shown and described with respect to FIG. 3a except that the etched electrodes 204 are replaced with coiled electrodes 204.

As with the coiled electrodes of FIG. 2a, the coiled electrodes 204 of FIG. 3b may be 0.005″ gauge (0.003″ to 0.006″ range with one preferred type being a 0.005″ gauge) platinum iridium wire that is wound into a spring-like structural unit. These units may be 3 mm to 6 mm long and have outer diameters ranging from approximately 3 Fr to 5 Fr. Other dimensions are also possible.

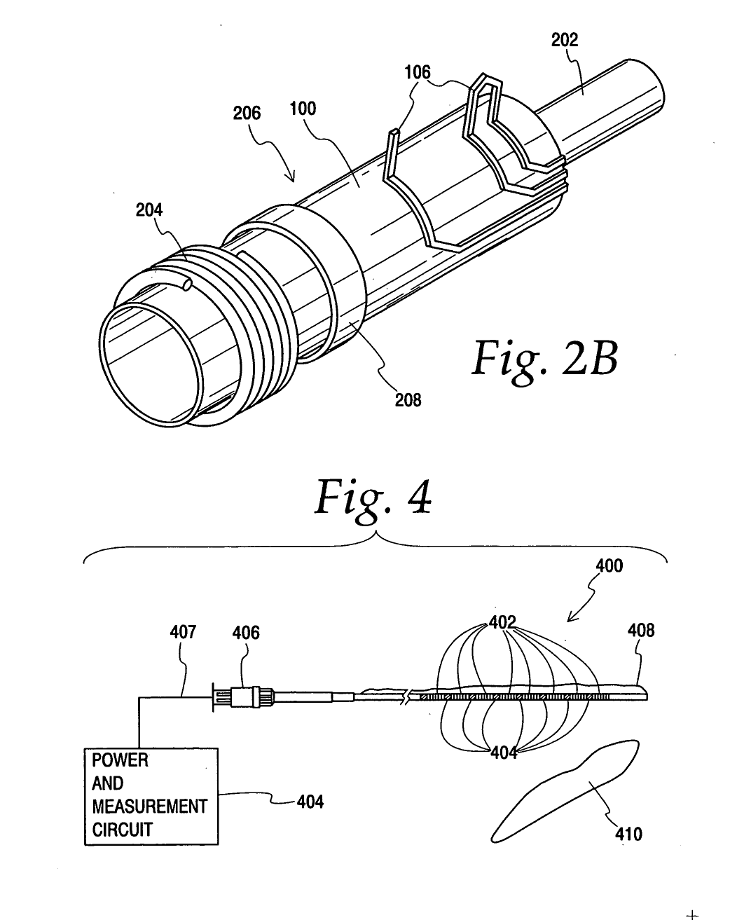

Referring now to FIG. 4, one example of a catheter system using the flex circuit and etched electrodes and thermocouples is described. A catheter 400 includes the cylindrical flex circuit assembly 408 that has been described with respect to FIGS. 1-3 above. The cylindrical assembly 408 forms the distal end of the catheter 400 and is inserted into the telescopic structure 406 having a handle, which forms the proximal end of the catheter 400.

Etched electrodes 402 are constructed and soldered onto the cylindrical assembly 408 as has been described elsewhere in the application. Alternatively, coiled electrodes may be used. In addition, thermocouples 404 are soldered onto the cylindrical assembly 408 as has also been described elsewhere in the application. The cylindrical assembly 408 may include sub-portions of flex circuits that are attached together to form the assembly 408.

An inner hollow shaft (not shown in FIG. 4) of the cylinder 408 (i.e., the flex circuit catheter shaft) may serve as a conduit for a guide wire or stylet with deflectable mechanism (not shown), permitting the linear assembly of electrodes 402 and thermocouples 404 to be shaped and conform to a tissue surface. This gives excellent electrode-tissue contact that ensures optimal coupling of RF energy with tissue 410. The conductive annular gold band for the thermocouples 404 and the etched electrode 402 may then be slid along the shaft and soldered over their respective solder points.

A power and measurement circuit 408 is coupled to the catheter 400 via a personal computer (PC) board 407. The power and measurement circuit 408 supplies electrical energy to the catheter and its electrodes 402 that can be used, for example, for ablation procedures. The impedance signals received at the electrodes and the information received by the thermocouples reporting tissue temperature can be relayed back to the power and measurement circuit 408 via the cylindrical assembly 408. The power and measurement circuit 408 can receive information from the thermocouples and display this information to an operator for manual feedback control. In addition, the power and measurement circuit 408 can receive operating instructions from an automated processing unit for feedback and control to adjust various operating parameters pertaining to the RF current being emitted from the catheter 400, such as the power or current delivered to the tissue 410.

Referring now to FIG. 5, a cross-sectional view of the cylindrical assembly 208 taken along line 304 in FIG. 3a is described. A guide wire 502 is in the middle of the hollow shaft 504 of the assembly 408. The electrodes 204 and thermocouple (not shown in FIG. 5) are soldered at the respective protruding pin sites 106 that were spaced at predetermined distances by design to provide the desired inter-electrode and electrode-thermocouple spacing along the side of the catheter.

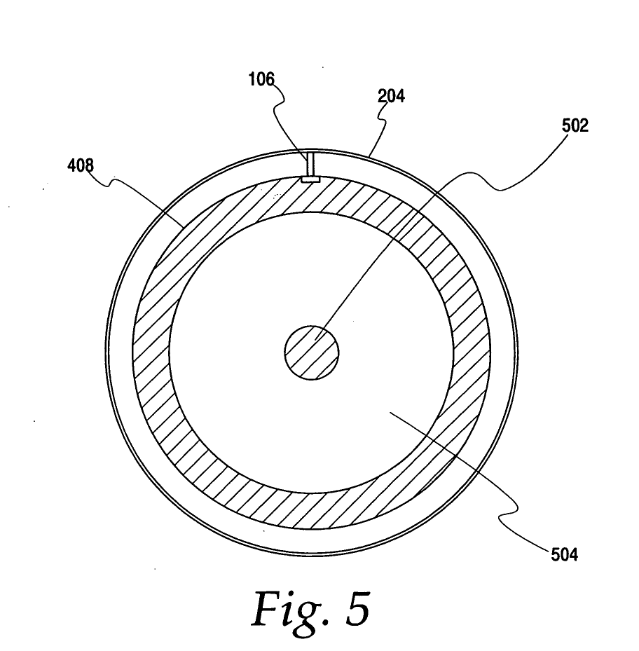

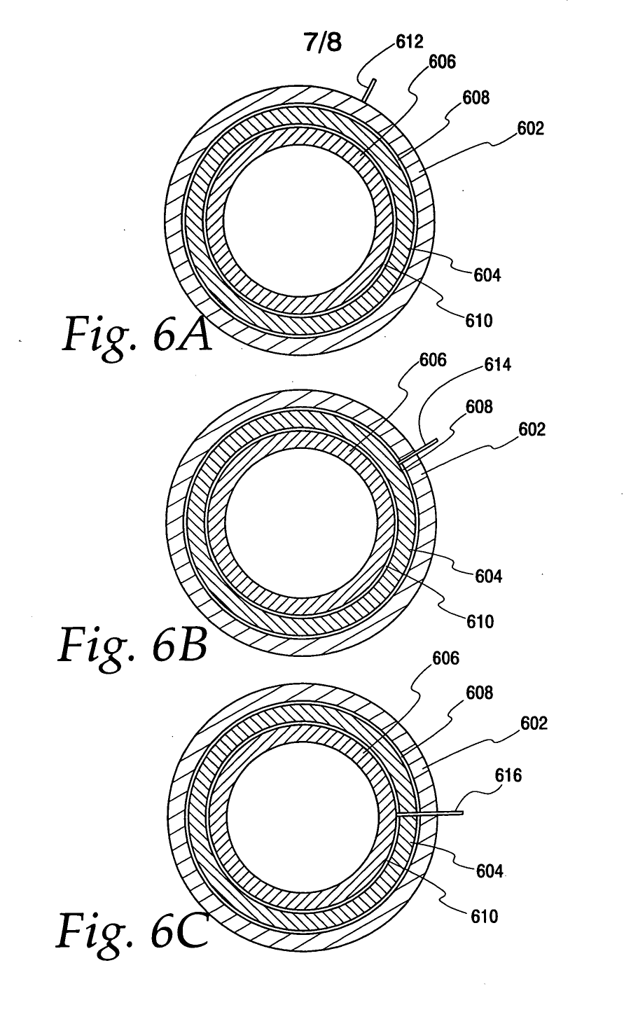

Referring now to FIG. 6a-c and FIG. 7, one example of an assembly using multiple layers of flex circuits is described. FIGS. 6a-c show cross sectional drawings taken along lines 708, 710, and 712 of FIG. 7 respectively. A first flex circuit assembly 602, second flex circuit assembly 604, and third flex circuit assembly 606 are concentrically located with assembly 602 on the outside, assembly 604 inside of assembly 602 and assembly 606 inside assembly 604.

The assemblies 602, 604, and 606 are electrically insulated from each other by a homogenous polyimide material layers 608 and 610 that is typically used in multi-layer flex circuit boards. Pin 612 is coupled to the flex circuit assembly 602. Pin 614 extends through the assembly 602 and is coupled to the flex circuit assembly 604. Pin 616 extends through the assemblies 602 and 604 and is coupled to the flex circuit assembly 606. Although only one pin is shown for each assembly (for convenience in viewing), it will be understood that multiple pins for the multiple layers 602, 604, and 606 can be used. In addition, additional pins for thermocouples may also be included. The inner pins 614 and 616 may have holes drilled through the various layers so that the pins 614 and 616 reach above the surface of the cylinder.

Referring now to specifically to FIG. 7, the assembly of FIG. 6 shows electrodes and thermocouples 702 coupled to the pins 612. Electrodes and thermocouples 704 are coupled to the pins 614. Further, electrodes and thermocouples 706 are coupled to the pins 616. Since multiple layers are used, the overall catheter or medical probe shaft diameter is reduced.

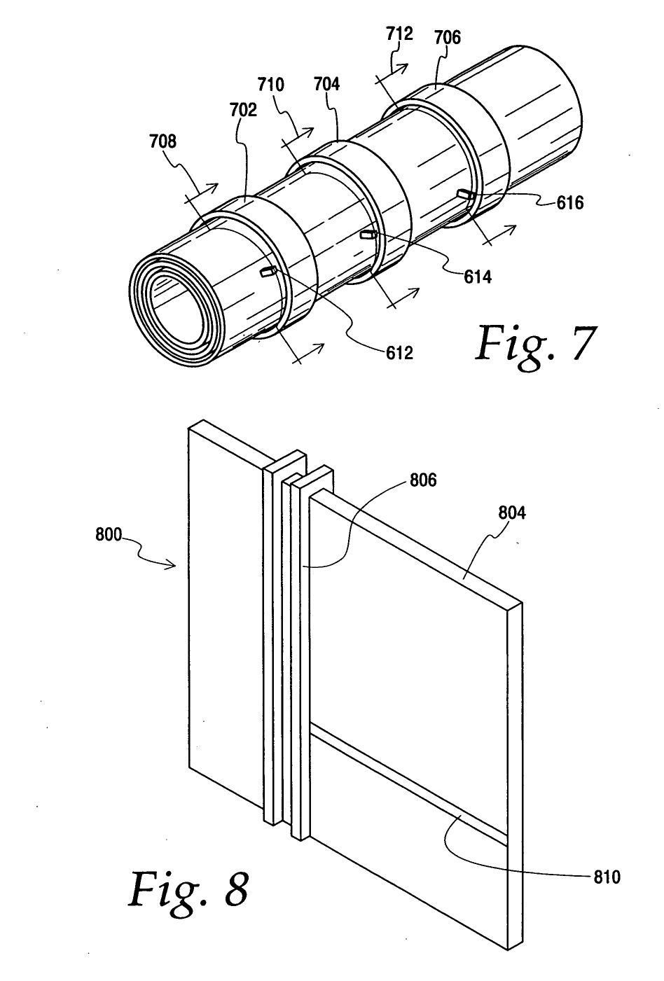

Referring now to FIG. 8, one example of a flex circuit 800 used in an ablation catheter is described where the electrodes are etched directly onto the flex sheet. A flex circuit pattern is printed on a flat sheet 804. Electrodes 806 are constructed on the sheet 804 directly and electrically contact a conductive circuit element 810 on the flex sheet 804. Dissimilar metals are layered onto the board, for instance, platinum for electrodes and copper for the conduction circuit element 810, by an appropriate manufacturing process.

Conductive circuit elements 810 of the sheet 804 are electrically insulated from each other and from the exposed surfaces of the flex sheet 804. Preferably, the inter-wire spacings for RF energy and current delivery are predetermined to comply with applicable regulatory, EMC and safety compliance standards.

Thus, the present system and method allows for the substitution of a flex circuit assembly for complex braided wire arrangements. It is simple to construct and incorporate into a catheter, surgical probe, or other medical device. Potentially, during the assembly process, a technician can easily replace damaged parts of the circuit with new flex circuit components as required. The etched electrodes provide for more precise dimensions to be provided for the electrodes than were possible in the previous arrangements.

While there has been illustrated and described particular embodiments of the present invention, it will be appreciated that numerous changes and modifications will occur to those skilled in the art, and it is intended in the appended claims to cover all those changes and modifications which fall within the true scope of the present invention.

What is claimed is:

1. A catheter having distal and proximal ends for performing ablation on a human tissue region comprising:
at least one electrode for performing ablation on human tissue formed from etching a conductive material and situated at a distal end of the catheter;
a flex circuit assembly coupling the at least one electrode to a measurement and power circuit attached to a proximal end of the catheter, the measurement and power circuit supplying power to the at least one electrode via the flex circuit.

2. The catheter of claim 1 further comprising at least one thermal sensing element that is formed from etching the conductive material and situated on the distal end of the catheter and wherein the at least one thermal sensing element supplies information indicative of thermal conditions of the contacting tissue region to the measurement and power circuit.

3. The catheter of claim 2 wherein the at least one electrode is capable of emitting energy to change tissue and wherein the at least one electrode provides information indicative of electrical conditions of a contacting tissue region to the measurement and power circuit.

4. The catheter of claim 1 wherein the at least one electrode is selected from a group comprising: an etched electrode and a coiled electrode.

5. The catheter of claim 1 wherein the flex circuit assembly comprises a plurality of identical flex circuit sub-portions.

6. The catheter of claim 5 wherein the sub-portions are formed into a cylindrical shape.

7. The catheter of claim 5 wherein the measurement and power circuit comprises a PC board, an energy source, and monitoring control circuits for energy delivery.

8. The catheter of claim 1 wherein the at least one electrode is coated with a conductive gel and an anti-coagular gel.

9. The catheter of claim 2 wherein the at least one thermal sensing element comprises gold bands and copper-constantan junctions.

10. The catheter of claim 1 wherein the flex assembly comprises conductive circuit elements that are insulated from each other.

11. The catheter of claim 1 wherein the assembly comprises multiple flex-circuit layers.

12. A method for constructing a catheter and using the catheter for ablation comprising:
forming a plurality of flex sheets, connecting the flex sheets, and rolling the connected flex sheets to form a cylinder assembly;
adhering pins along a surface of the cylinder assembly, the pins coupled to flex circuitry positioned on the flex sheets; and
coupling electrodes to the pins; and
transmitting energy through the flex circuitry of the flex sheets to the electrodes in order to perform ablation on human tissue.

13. The method of claim 12 wherein coupling electrodes comprises coupling electrodes selected from a group comprising: etched electrodes and coiled electrodes.

14. The method of claim 12 further comprising coupling at least one thermocouple banding along the surface of the cylinder assembly.

15. The method of claim 14 further comprising attaching a PC board connection at a proximal end of the cylinder and attaching a PC board to a power and measurement and control circuit.

16. The method of claim 12 further comprising covering the electrodes with a conductive gel.

17. The method of claim 12 further comprising infusing the electrodes with anti-coagulant chemicals that are time released during the course of an ablation procedure.

18. A catheter having distal and proximal ends for performing medical procedures on a human tissue region comprising:
at least one etched electrode and at least one thermocouple sensor situated at the distal end of the catheter;
a flex circuit assembly comprising identical multiple flex sheet portions and coupling the at least one etched electrode and the at least one thermocouple sensor to a measurement and power circuit attached to the proximal end of the catheter, the measurement and power circuit supplying power to the at least one etched electrode via the flex circuit to perform ablation, the assembly formed into a cylinder; and
wherein the at least one thermocouple sensor supplies information indicative of conditions of the human tissue region to the measurement and power circuit.

19. The catheter of claim 18 wherein the measurement and power circuit comprises a PC board, an energy source, and monitoring and control circuits for energy delivery.

20. The catheter of claim 18 wherein the medical procedures comprise ablation procedures.
